SEQUENCE LISTING (28 sequences)

SEQ ID NO: 1 - DNA, 783 bp, Artificial Sequence
  ISF30
  misc_feature (1)..(783)
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 2 - DNA, 759 bp, Artificial Sequence
  ISF31
  misc_feature (1)..(759)
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 3 - DNA, 783 bp, Artificial Sequence
  ISF32
  misc_feature (1)..(783)
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 4 - DNA, 759 bp, Artificial Sequence
  ISF33
  misc_feature (1)..(759)
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 5 - DNA, 783 bp, Artificial Sequence
  ISF34
  misc_feature (1)..(783)
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 6 - DNA, 759 bp, Artificial Sequence
  ISF35 (MEM40)
  misc_feature (1)..(759)
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 7 - DNA, 783 bp, Artificial Sequence
  ISF36
  misc_feature (1)..(783)
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcgtgttttgtcaatgtgactgatccaagccaagtgagccatggcactggcttcacgtcctttggcttactcaaactctga

SEQ ID NO: 8 - DNA, 759 bp, Artificial Sequence
  ISF37
  misc_feature (1)..(759)
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 9 - DNA, 783 bp, Artificial Sequence
  ISF38
  misc_feature (1)..(783)
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 10 - DNA, 759 bp, Artificial Sequence
  ISF39
  misc_feature (1)..(759)
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 11 - DNA, 783 bp, Artificial Sequence
  ISF40
  misc_feature (1)..(783)
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 12 - DNA, 759 bp, Artificial Sequence
  ISF41
  misc_feature (1)..(759)
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 13 - protein, 260 aa, Artificial Sequence
  Chimeric human/mouse CD40 ligand
  PEPTIDE (1)..(260)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSVHLGGVFELQPGASVFVNVTDPSQVSHGTGFTSFGLLKL

SEQ ID NO: 14 - protein, 252 aa, Artificial Sequence
  ISF31
  PEPTIDE (1)..(252)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSVHLGGVFELQPGASVFVNVTDPSQVSHGTGFTSFGLLKL

SEQ ID NO: 15 - protein, 260 aa, Artificial Sequence
  ISF32
  PEPTIDE (1)..(260)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSVHLGGVFELQPGASVFVNVTDPSQVSHGTGFTSFGLLKL

SEQ ID NO: 16 - protein, 252 aa, Artificial Sequence
  ISF33
  PEPTIDE (1)..(252)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSVHLGGVFELQPGASVFVNVTDPSQVSHGTGFTSFGLLKL

SEQ ID NO: 17 - protein, 260 aa, Artificial Sequence
  Artificial Sequence
  PEPTIDE (1)..(260)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 18 - protein, 252 aa, Artificial Sequence
  ISF35
  PEPTIDE (1)..(252)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 19 - protein, 260 aa, Artificial Sequence
  ISF36
  PEPTIDE (1)..(260)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSAKPCGQQSIHLGGVFELQPGASCFVNVTDPSQVSHGTGFTSFGLLKL

SEQ ID NO: 20 - protein, 252 aa, Artificial Sequence
  ISF37
  PEPTIDE (1)..(252)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSAKPCGQQSIHLGGVFELQPGASVFVNVTDPSQVSHGTGFTSFGLLKL

SEQ ID NO: 21 - protein, 260 aa, Artificial Sequence
  ISF38
  PEPTIDE (1)..(260)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSIHLGGVFELQPGASVFVNVTDPSQVSHGTGFTSFGLLKL

SEQ ID NO: 22 - protein, 252 aa, Artificial Sequence
  ISF39
  PEPTIDE (1)..(252)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSIHLGGVFELQPGASVFVNVTDPSQVSHGTGFTSFGLLKL

SEQ ID NO: 23 - protein, 260 aa, Artificial Sequence
  ISF40
  PEPTIDE (1)..(260)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSAKPCGQQSIHLGGVFELQPGASVFVNVTDPSQVSHGTGFTSFGLLKL

SEQ ID NO: 24 - protein, 252 aa, Artificial Sequence
  ISF41
  PEPTIDE (1)..(252)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSAKPCGQQSIHLGGVFELQPGASVFVNVTDPSQVSHGTGFTSFGLLKL

SEQ ID NO: 25 - DNA, 3768 bp, SARS-CoV-1
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 26 - protein, 1273 aa, SARS-CoV-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - DNA, 3822 bp, SARS-CoV-2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 28 - protein, 1273 aa, SARS-CoV-2; identical to SEQ ID NO: 26

(19) United States
(12) Patent Application Publication
(10) Pub. No.: US 2022/0090134 A1
(43) Pub. Date: Mar. 24, 2022

(54) Enhancing Immunity Using Chimeric CD40 Ligand and Coronavirus Vaccine

(71) Applicant: Memgen, Inc., Houston, TX (US)

(72) Inventor: Mark J. CANTWELL, Meadow Vista, CA (US)

(73) Assignee: Memgen, Inc., Houston, TX (US)

(21) Appl. No.: 17/472,268

(22) Filed: Sep. 10, 2021

Related U.S. Application Data
(60) Provisional application No. 63/077,204, filed on Sep. 11, 2020.

(51) Int. Cl. C12N 15/86; A61K 39/215; A61P 31/14; A61K 39/39; C12N 7/00

(52) U.S. Cl. CPC C12N 15/86; A61K 39/215; A61K 2039/5252; A61K 39/39; C12N 7/00; A61P 31/14

(57) ABSTRACT

The present disclosure provides methods and compositions for enhancing immunity by administering a coronavirus vaccine and a chimeric CD40L polypeptide. The coronavirus vaccine can be comprised of inactivated coronaviral particles or an antigenic polypeptide, preferably the coronavirus spike protein. The coronavirus antigenic polypeptide can be a purified antigenic polypeptide or a nucleic acid expression construct that encodes the antigenic polypeptide. The chimeric CD40L polypeptide in compositions of the invention can be a purified chimeric CD40L polypeptide or a nucleic acid expression construction that encodes the chimeric CD40L polypeptide.

### CROSS-REFERENCE TO RELATED APPLICATION

This application claims the benefit of Provisional Application Ser. No. 63/077,204, filed Sep. 11, 2020, which is herein incorporated by reference.

### BACKGROUND OF THE INVENTION

### 1. Field of the Invention

The present invention relates generally to vaccines and vaccine adjuvants and methods for enhancing immunity against infectious agents. In particular, the present invention relates to the use of chimeric CD40 ligand (CD40L) as a vaccine adjuvant, specifically with respect to a coronavirus vaccine.

### 2. Description of Related Art

The body defends against infectious pathogens and microorganisms using both the innate and adaptive immune systems. The innate immune system generally operates as the first line of defense through key effector cells such as neutrophils, macrophages, and natural killer cells through their recognition and response against characteristic structures found on pathogens generally not present on mammalian cells. These pathogenic structures operate as signals and are called damage-associated molecular patterns (DAMPs) and pathogen-associated molecular patterns (PAMPs). The second line of defense through the adaptive immune system is mediated primarily by B cells and T cells that make up humoral and cell-mediated immunity, respectively. The adaptive immune system can evolve to specifically target and eliminate a specific pathogen, as well as provide longer-term surveillance and response upon pathogen re-challenge or attack.

A vaccine is a product that stimulates a person's immune system to produce immunity to a specific disease, protecting the person from that disease. A person or animal may be administered a vaccine that stimulates their immune system to produce humoral (antibodies) and/or cellular (T cells) immune responses to one or more antigens present on the pathogen to try to protect against diseases arising from pathogenic infection. This primes the immune system so that when the body is exposed to the pathogen in the future, memory cells of the adaptive immune system will recognize it, and the body's response to eliminate the pathogen will be much stronger. Typical vaccines are comprised of inactivated or attenuated virus particles, antigenic polypeptides, or genetic constructs encoding for antigenic polypeptides. One specific area of recent vaccine development has been with respect to coronaviruses, including, for example, SARS-CoV-1 and SARs-CoV-2.

Many vaccines show promise in eliciting an immune response but not a sufficient response to be fully protective against the disease. For this reason, some vaccines are combined with adjuvants. Adjuvants may be co-administered with a vaccine to create a stronger immune response in a person or animal receiving the vaccine.

All of the subject matter discussed in the Background is not necessarily prior art and should not be assumed to be prior art merely as a result of its discussion in the Background section. Along these lines, any recognition of problems in the prior art discussed in the Background or associated with such subject matter should not be treated as prior art unless expressly stated to be prior art. Instead, the discussion of any subject matter in the Background should be treated as part of the inventor's approach to the particular problem, which in and of itself, may also be inventive.

### SUMMARY OF THE INVENTION

The following presents a simplified summary of the disclosure in order to provide a basic understanding of some aspects of the disclosure. This summary is not an exhaustive overview of the disclosure. It is not intended to identify key or critical elements of the disclosure or to delineate the scope of the disclosure. Its sole purpose is to present some concepts in a simplified form as a prelude to the more detailed description that is discussed later.

The present disclosure overcomes several major problems associated with current technologies by providing methods and compositions for enhancing immunity by administering a coronavirus vaccine and a chimeric CD40L polypeptide. The chimeric CD40L polypeptide can be provided as a polypeptide or via administration of an expression construct from which the chimeric CD40L can be expressed. Specifically, it is contemplated that an expression construct for the chimeric CD40L polypeptide can be a viral vector, such as an adenoviral construct that includes a eukaryotic transcriptional promoter operably linked to a protein-coding sequence of a chimeric CD40L and a transcriptional termination sequence. As detailed below, chimeric CD40L acts as an adjuvant to enhance immunity to coronavirus when administered with a coronavirus vaccine. This invention contemplates that the vaccine against coronavirus may comprise inactivated coronaviral particles or an antigenic polypeptide, preferably the coronavirus spike protein. In addition, this invention contemplates the coronavirus vaccine may comprise coronavirus antigen administered via an expression construct, which can be a viral vector, such as an adenoviral construct that includes a eukaryotic transcriptional promoter operably linked to a protein-coding sequence of a coronavirus antigen, preferably coronavirus spike protein, and a transcriptional termination sequence. In certain embodiments, the chimeric CD40L polypeptide and coronavirus antigen are expressed from the same expression construct.

A chimeric CD40L polypeptide includes at least one subdomain from two different species. In certain embodiments, the chimeric CD40L polypeptide includes domains and/or subdomains from both human CD40L and murine CD40L. For example, the chimeric CD40L polypeptide is selected from the group consisting of ISF30, ISF31, ISF32, ISF33, ISF34, ISF35, ISF36, ISF37, ISF38, ISF39, ISF40, and ISF41. In particular, the chimeric CD40L polypeptide is ISF35.

In further aspects, the chimeric CD40L polypeptide and/or coronavirus antigen is administered to a subject by providing a coding region for the chimeric CD40L polypeptide and/or coronavirus antigen in an expression vector and under control of a promoter(s) active in a eukaryotic cell under conditions supporting expression of said chimeric CD40L polypeptide and, as applicable, coronavirus antigen. In some aspects, the expression cassette is in a viral vector. For example, the viral vector is an adenoviral vector, a retroviral vector, a coronaviral vector, a pox viral vector, a herpes viral vector, an adeno-associated viral vector, or a polyoma viral vector. In particular, the viral vector is an adenoviral vector.

The details of one or more embodiments are set forth in the description below. The features illustrated or described in connection with one exemplary embodiment may be combined with the features of other embodiments. Thus, any of the various embodiments can be modified, if necessary, to employ concepts of the various patents, applications, and publications as identified herein to provide yet further embodiments. Other features, objects and advantages will be apparent from the description, the drawings, and the claims.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. shows a diagram of an adenoviral expression construct encoding a chimeric CD40L polypeptide.

FIG. 2. shows a diagram of an adenoviral expression construct encoding a chimeric CD40L polypeptide and a coronavirus antigen.

FIG. 3. shows the number of coronavirus spike protein antigen-specific T cells as measured by ELISPOT IFNγ secretion.

FIG. 4. shows the coronavirus spike protein-specific antibody (IgG) humoral immune response as measured by ELISA

### BRIEF DESCRIPTION OF THE SEQUENCES

SEQ ID NOs: 1-12 are nucleic acid sequences encoding chimeric human/mouse CD40L (the chimeric human/mouse CD40L encoded by these sequences referred to, respectively, as ISF30, ISF31, ISF32, ISF33, ISF34, ISF35, ISF36, ISF37, ISF38, ISF39, ISF40, and ISF41).

SEQ ID NOs: 13-24 are examples of chimeric CD40L amino acid sequences (ISF30, ISF31, ISF32, ISF33, ISF34, ISF35, ISF36, ISF37, ISF38, ISF39, ISF40, and ISF41, respectively).

SEQ ID NO. 25 is the nucleic acid sequence encoding the coronavirus spike protein of SARS-CoV-1.

SEQ ID NO. 26 is the amino acid sequence for the coronavirus spike protein of SARS-CoV-1.

SEQ ID NO. 27 is the nucleic acid sequence encoding the coronavirus spike protein of SARS-CoV-2.

SEQ ID NO. 28 is the amino acid sequence for the coronavirus spike protein of SARS-CoV-2.

### DETAILED DESCRIPTION OF THE INVENTION

Various illustrative embodiments of the disclosure are described below. In the interest of clarity, exemplary embodiments in this specification do not describe all features of an actual implementation. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex but would nevertheless be a routine undertaking for those of ordinary skill in the art having the benefit of this disclosure.

The present disclosure overcomes several major problems associated with current technologies by providing methods and compositions for enhancing function of an immune cell by providing a combination of at least one coronavirus vaccine in combination with a chimeric CD40L polypeptide or nucleic acid encoding a chimeric CD40L polypeptide.

In certain embodiments of the methods of the present disclosure, the chimeric CD40L polypeptide comprises a non-human CD40L cleavage site and an extracellular subdomain of human CD40L that binds to a human CD40 receptor. For example, an extracellular subdomain of human CD40L which comprises a cleavage site is replaced by an extracellular subdomain of non-human CD40L, such as murine CD40L. In particular embodiments, the chimeric CD40L polypeptide is ISF35. In one method, the chimeric CD40L polypeptide is delivered in an expression cassette, such as an adenoviral vector, encoding the polypeptide, in particular under the control of a promoter active in a eukaryotic cell. In other methods, both the chimeric CD40L polypeptide and coronavirus antigen, such as coronavirus spike protein, are delivered in an expression cassette, such as an adenoviral vector, encoding the polypeptides, in particular under the control of one or more promoters active in a eukaryotic cell.

### A. Definitions

As used in this specification, “a” or “an” may mean one or more. As used in the claim(s), when used in conjunction with the word “comprising,” the words “a” or “an” may mean one or more than one.

Throughout this application, the term “about” is used to indicate that a value includes the inherent variation of error for the device, the method being employed to determine the value, or the variation that exists among the study subjects.

The term “antibody” herein is used in the broadest sense and specifically covers monoclonal antibodies (including full length monoclonal antibodies), polyclonal antibodies, multi-specific antibodies (e.g., bispecific antibodies), and antibody fragments so long as they exhibit the desired biological activity.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients may also be incorporated into the compositions.

As used herein, the terms “CD40 ligand”, “CD40L” and “CD154” are used interchangeably herein. For example, an adenoviral construct encoding a chimeric CD40 ligand may be referred to as ad-CD40L.

The term “chimeric” is defined as having sequences from at least two different species.

The terms “chimeric CD40L” or “chimeric ISF construct” refers to a ligand comprised of at least one domain or subdomain of CD40L from one species and at least one domain or subdomain of CD40L from a different species. In certain embodiments, the at least two species from which the chimeric CD40L is derived are human and murine CD40L.

As used herein, the term “cleavage site” refers to a sequence of amino acids that is recognized by proteases, typically matrix metalloproteases (MMP) that cleave CD40L from the surface of the expressing cell. The cleavage site of CD40L is typically found at or around the boundaries of domains III and IV of CD40L. For example, one such cleavage site comprises the region approximately between amino acids 108 and 116 of human CD40L.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “coronavirus antigen” refers to a polypeptide that induces an immune response to coronavirus infection, for example, the coronavirus spike protein. SEQ ID NO. 26 provides the amino acid sequence for the coronavirus spike protein of SARs-CoV-1. SEQ ID No. 28 provides the amino acid sequence for the coronavirus spike protein of SARs-CoV-2. As used herein, “coronavirus spike protein” refers to a polypeptide that is substantially homologous to either SEQ ID NO. 26 or SEQ ID NO. 28.

The term “coronavirus vaccine” refers to a vaccine that contains either inactivated or attenuated coronaviral particles, a coronavirus antigen, or an expression construct that encodes a coronavirus antigen.

As used herein, the term “corresponding” refers to the sequence of nucleotides or amino acids of CD40L of one species that is substantially homologous to a nucleotide or amino acid sequence of CD40L of another species. This homology is based on the similarity in secondary structure, such as the location of domain boundaries, among CD40L of different species.

An “effective amount” is at least the minimum amount required to effect a measurable improvement or prevention of a particular disease. An effective amount herein may vary according to factors such as the disease state, age, sex, and weight of the patient, and the ability of the vaccine and/or adjuvant to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “enhancer” means a nucleic acid sequence that, when positioned proximate to a promoter, confers increased transcription activity relative to the transcription activity resulting from the promoter in the absence of the enhancer sequence.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. For example, an exogenous nucleic acid may be one that is in a chromosomal location different from where it would be in natural cells, or is otherwise flanked by a different nucleic acid sequence than that found in nature.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

“Homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, in particular at least about 90%, and most particularly at least about 95% of the nucleotides, or amino acids, respectively match over a defined length of the molecules, as determined using the methods above.

As used herein, the phrases “less susceptible to cleavage” or “reduced cleavage” refer to the higher resistance of a chimeric CD40L to proteolytic cleavage compared to that of native human CD40L, as measured by the amount of soluble CD40L generated by a given number of cells over a period of time. In particular, a chimeric CD40L of the present invention is “less susceptible to cleavage” because it is cleaved at a rate at least 50%, at least 75%, or at least 90% less than that of native CD40L.

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g., adenine “A,” guanine “G,” thymine “T,” and cytosine “C”) or RNA (e.g. A, G, uracil “U,” and C). The term “nucleic acid” encompasses the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

By “operably linked” or “co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is in particular chimeric, i.e., composed of heterologous molecules.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “plasmid,” a common type of a vector, is an extra-chromosomal DNA molecule separate from the chromosomal DNA that is capable of replicating independently of the chromosomal DNA. In certain cases, it is circular and double-stranded.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control” and “under transcriptional control” mean that a promoter is in a correct functional location and/or orientation in relation to a nucleic acid sequence to control transcriptional initiation and/or expression of that sequence.

The term “subdomain” refers to a sequence of at least two amino acids that is part of a domain of CD40L. A “subdomain” also encompasses an amino acid sequence from which one or more amino acids have been deleted, added, or has been modified, including one or more amino acids truncated from an end of the sequence.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

### B. CD40 and CD40 Ligand

CD40 is a 50 Kd glycoprotein expressed on the surface of B cells, dendritic cells, normal epithelium and some epithelial carcinomas (Briscoe et al, 1998). The ligand for CD40, CD40L, is expressed on activated T lymphocytes, human dendritic cells, human vascular endothelial cells, smooth muscle cells, and macrophages. CD40L exists on such cells as a trimeric structure, which induces oligomerization of its receptor upon binding.

CD40 ligand (also known as CD40L, gp39, or CD154) is a type II membrane polypeptide having an extracellular region at its C-terminus, a transmembrane region and an intracellular region at its N-terminus. The CD40 ligand has been cloned and sequenced, and nucleic acid and amino acid sequences have been reported from human (GenBank accession numbers Z15017/S49392, D31793-7, X96710, L07414 and X67878/S50586), murine (GenBank accession number X65453), bovine (GenBank accession number Z48469), canine (GenBank accession number AF086711), feline (GenBank accession number AF079105) and rat (GenBank Accession Numbers AF116582, AF013985). Such murine, bovine, canine, feline and rat sequences are also disclosed in U.S. Pat. No. 6,482,411, incorporated herein by reference. Additional CD40 ligand nucleic acid and amino acid sequences are disclosed in U.S. Pat. Nos. 5,565,321 and 5,540,926, incorporated herein by reference, and mutant/chimeric CD40 ligand sequences are disclosed in U.S. Pat. Nos. 5,716,805; 5,962,406; 6,087,329; 7,495,090; 7,524,944; 7,928,213; and 8,138,310, each of which is incorporated herein by reference.

### C. Chimeric CD40L Polypeptides

CD40L is one member of a larger family of ligands, collectively referred to as the TNF superfamily (Gruss et al, Cytokines Mol Ther, 1:75-105, 1995 and Locksley et al, Cell, 104:487-501, 2001). Members of the TNF superfamily include Fas ligand (“FasL”), TNFα, LTα, lymphotoxin (TNFβ), CD154, TRAIL, CD70, CD30 ligand, 4-1BB ligand, APRIL, TWEAK, RANK ligand, LIGHT, AITR ligand, ectodysplasin, BLYS, VEGI, and OX40 ligand. TNF superfamily members share a conserved secondary structure comprising four domains: domain I, the intracellular domain; domain II, which spans the cell membrane and is known as the transmembrane domain; domain III, which consists of the extracellular amino acids closest to the cell membrane; and domain IV, the distal extracellular domain. Typically, at least a part of domain IV can be cleaved from the parent molecule. The cleaved fragment often exhibits the same biological activity of the intact ligand and is conventionally referred to as a “soluble form” of the TNF family member. Soluble versions of CD40 ligand can be made from the extracellular region, or a fragment thereof, and a soluble CD40 ligand has been found in culture supernatants from cells that express a membrane-bound version of CD40 ligand, such as EL-4 cells.

The interactions between CD40L and its cognate receptor, CD40, are critical for immune recognition. (Banchereau J. et al., Annu. Rev. Immunol. 12:881-922, 1994). CD40L is transiently expressed on CD4⁺ T cells following T cell receptor engagement by antigen presenting cells through MEW class II molecules (Cantwell M. et al., Nat. Med., 3:984-989, 1997). This, in turn, can cause activation of CD40-expressing antigen presenting cells (APCs), including B cells, dendritic cells, monocytes, and macrophages (Ranheim E. A. et al., Cell. Immunol., 161:226-235, 1995). Such CD40 activated cells can set off a cascade of immune-activating events that lead to a specific and effective immune response against foreign antigens, such as viruses or tumors.

It is known in the art that at least part of human CD40L is cleaved from the parent molecule and becomes a soluble molecule, however, the soluble form is generally undesirable. Thus, the chimeric CD40L polypeptide of the present disclosure can be formed by exchanging an amino acid, or an amino acid sequence, of human CD40L that comprises a cleavage site recognized by proteolytic enzymes with an amino acid, or amino acid sequence, of non-human CD40L, that does not contain this cleavage site. In certain embodiments, the non-human CD40L is murine CD40L. Alternatively, the chimeric CD40L polypeptide can include a point mutation at the cleavage site or deletion of the cleavage site.

In some embodiments, the chimeric CD40L polynucleotide sequence comprises a first nucleotide sequence encoding an extracellular subdomain of non-human CD40L that corresponds to and replaces a cleavage site of human CD40L. The chimeric CD40L polypeptide can be produced by replacing a subdomain of human CD40L containing a CD40L cleavage site with the corresponding subdomain of non-human CD40L results in a chimeric CD40L that is markedly less susceptible to cleavage than human CD40L. In other embodiments, the amino acids of the cleavage site are modified, altered or deleted to decrease susceptibility to cleavage by a protease. The first nucleotide sequence can be operatively linked to a second nucleotide sequence that encodes an extracellular subdomain of human CD40L involved in binding to a human CD40 receptor. In this way, the polynucleotide sequence of the present disclosure encodes a chimeric CD40L that binds to human cells expressing the CD40 receptor. Moreover, in some aspects, an extracellular domain of murine and human CD40L includes at least one amino acid, or a sequence of amino acids, that allows expression of the molecule on the membranes of murine and human cells.

The CD40L polypeptides of the present disclosure may be chimeric in that they can be comprised of CD40L domains or subdomains from at least two different species, in some cases human and mouse CD40L. These polypeptides are designated “immune stimulatory factors”, or ISFs, because they combine human and non-human CD40L regions to maximize stimulation of the immune response. Specifically, at least one domain or subdomain of CD40L that contains a cleavage site of human CD40L is replaced with a corresponding domain or subdomain of non-human CD40L, in particular murine CD40L. In addition, the chimeric polypeptide is composed of a domain or subdomain of human CD40L that is responsible for binding a CD40L receptor.

Chimeric CD154 or CD40L polypeptides for use in the present disclosure are described in U.S. Pat. Nos. 7,495,090 and 7,928,213, both incorporated herein by reference. For example, domain IV of human CD40L can be linked to domains I, II and III of murine CD40L. Examples of such in particular polynucleotide sequences are provided herein as SEQ ID. NOS. 1, 3, 5, 7, 9 and 11 and encode chimeric CD40L constructs designated as ISF 30, 32, 34, 36, 38 and 40, respectively. Additionally, domain IV of murine CD40L may be linked to domains I, II and III of human CD40L. Examples of such polynucleotide sequences are provided as SEQ ID. NOS. 2, 4, 6, 8, 10 and 12, and encode chimeric CD40L constructs are designated ISF 31, 33, 35, 37, 39 and 41, respectively. In a particular embodiment, the chimeric CD40L polypeptide used in the invention is ISF35.

### D. Methods of Polypeptide Delivery

In some embodiments, the chimeric CD40L polypeptide and/or coronavirus antigen is delivered in nanoparticles. For example, the nanoparticles are made of biodegradable polymers such as poly lactic acid, polycaprolactone, poly(lactic-co-glycolic acid), the poly(fumaric-co-sebacic) anhydride chitosan, and modified chitosan. Alternatively, the chimeric CD40L polypeptide and/or coronavirus antigen is delivered in liposomes, PEGylated liposomes, niosomes, or aquasomes. Other methods known in the art for peptide or protein delivery may be used such as described in U.S. Pat. Nos. 8,288,113 and 5,641,670, and U.S. Patent Publication Nos. US20100291065, US20140242107, US2014023213, US20150191710, and US2010026678; all of which are incorporated herein by reference.

In some embodiments, the chimeric CD40L polypeptide and/or coronavirus antigen are provided in an expression construct. In particular, the chimeric CD40L construct would be membrane-stabilized and resistant to proteolytic cleavage, and thereby less likely to generate the soluble form of CD40L. However, the chimeric CD40L construct would maintain the receptor-binding function of native CD40L. Moreover, a particular CD40L construct would not be immunogenic at the domain critical for receptor binding following administration in humans, thus avoiding functional neutralization.

One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc.), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, coronavirus vectors, and Rous sarcoma virus vectors.

1. Viral Vectors

Viral vectors encoding the chimeric CD40L polypeptide and/or coronavirus antigen may be provided in certain aspects of the present invention. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

a. Lentiviral Vector

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

b. Adenoviral Vector

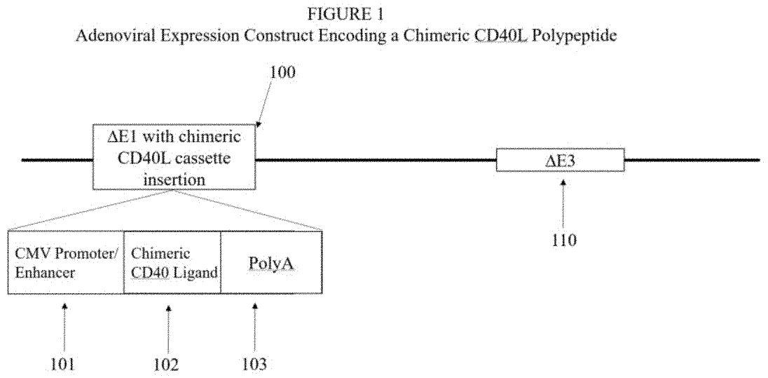

Another example of a viral vector is an adenovirus expression vector, which can be used as a method for delivery of the chimeric CD40L polypeptide and/or the coronavirus antigen. Although adenovirus vectors are known to have a low capacity for integration into genomic DNA, this feature is counterbalanced by the high efficiency of gene transfer afforded by these vectors. Adenovirus expression vectors include constructs containing adenovirus sequences sufficient to (a) support packaging of the construct and (b) to ultimately express a recombinant gene construct that has been cloned therein. FIG. 1 shows a diagram of an adenoviral expression construct that encodes a chimeric CD40L polypeptide. In certain embodiments, the chimeric CD40L sequence 102 used in the expression construct is chosen from one of a sequence encoding ISF31, ISF32, ISF33, ISF34, ISF35, ISF36, ISF37, ISF38, ISF39, ISF40, ISF41, or ISF42, preferably ISF35, or is substantially homologous to one of the foregoing sequences. The transcription of the chimeric CD40L is controlled by inclusion additional regulatory regions, including a promoter/enhancer region 101, typically upstream of the chimeric CD40 ligand sequence 102, and a polyadenylation sequence 103, typically downstream of the CD40 ligand sequence. Although FIG. 1 shows the CMV promoter 101, another promoter, such as those disclosed herein, could be used in the expression construct so long as it promotes expression of the chimeric CD40L polypeptide. Although FIG. 1 shows the chimeric CD40L cassette inserted into the adenoviral genome at the E1 deletion site, alternative insertion sites into the adenoviral genome for the chimeric CD40L expression cassette could be utilized so long as it promotes expression of the chimeric CD40L polypeptide. In the adenoviral expression construct depicted in FIG. 1, the E1 region of the adenovirus genome was deleted and replaced with the chimeric CD40L cassette insertion 100 and the E3 region of the adenovirus genome 110 was deleted.

MemVax contains an adenoviral expression vector that encodes a chimeric CD40L polypeptide. Specifically, the adenoviral expression vector in MemVax encodes ISF35. MemVax also includes a storage formulation comprising a TRIS-lactose buffered solution allowing for both storage and administration to humans or animals across multiple routes of administration, including by injection, intranasal, or oral.

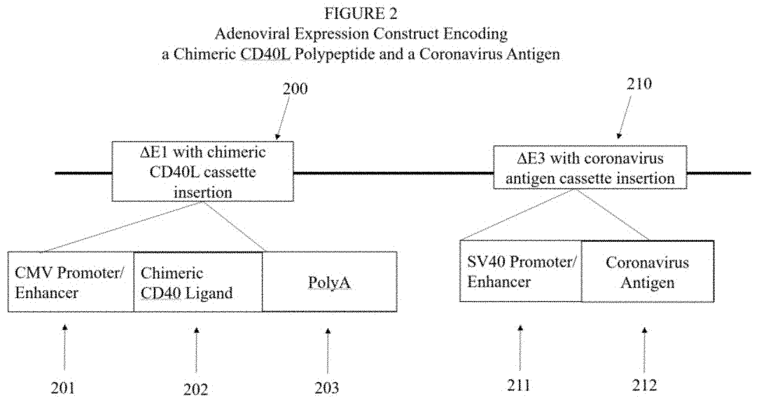

FIG. 2 shows a diagram of a combined adenoviral expression construct that encodes both a chimeric CD40L polypeptide and a coronavirus antigen. Preferably, the chimeric CD40L sequence 202 used in the combined expression construct is chosen from one of a sequence encoding ISF30, ISF31, ISF32, ISF33, ISF34, ISF35, ISF36, ISF37, ISF38, ISF39, ISF40, or ISF41, preferably ISF35, or is substantially homologous to one of those sequences. Preferably, the coronavirus antigen sequence 212 used in the combined expression construct is chosen from one of a sequence encoding the coronavirus spike protein of SARS-CoV-1 or SARS-CoV-2. The transcription of the both the chimeric CD40L and coronavirus antigen are controlled by inclusion additional regulatory regions, including a promoter/enhancer region, typically upstream of the chimeric CD40 ligand sequence and the coronavirus antigen sequence, 201 and 211 respectively. Although FIG. 2 shows the CMV promoter for the chimeric CD40L 201 and the SV40 promoter for the coronavirus antigen 211, other promoters, such as those disclosed herein, could be used in the expression construct so long as it promotes expression of the chimeric CD40L polypeptide and the coronavirus antigen. In preferred embodiments, different promoters are used for chimeric CD40L and coronavirus antigen to reduce the risk of homologous recombination of the viral construct. FIG. 2 also depicts a polyadenylation sequence 203 in the chimeric CD40L cassette insertion 200. In FIG. 2, the chimeric CD40L cassette insertion 200 was inserted into the adenoviral genome at the E1 deletion site, and the coronavirus antigen cassette 210 was inserted into the adenoviral genome at the E3 deletion site. Although FIG. 2 shows the chimeric CD40L cassette insertion 200 was inserted into the adenoviral genome at the E1 deletion site, and the coronavirus antigen cassette 210 was inserted into the adenoviral genome at the E3 deletion site CMV promoter 101, alternative insertion sites into the adenoviral genome for each expression cassette could be utilized so long as it promotes expression of the chimeric CD40L polypeptide and the coronavirus antigen.

Adenovirus growth and manipulation is known to those of skill in the art, and exhibits broad host range in vitro and in vivo. This group of viruses can be obtained in high titers, e.g., 109-1011 plaque-forming units (pfus) per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′-tripartite leader (TPL) sequence which makes them particularly efficient mRNAs for translation.

A recombinant adenovirus can be generated from homologous recombination between a shuttle vector and provirus vector. Due to the possible recombination between two proviral vectors, wild-type adenovirus may be generated from this process. Therefore, a single clone of virus is isolated from an individual plaque and its genomic structure is examined.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the particular starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Nucleic acids can be introduced to adenoviral vectors as a position from which a coding sequence has been removed. For example, a replication defective adenoviral vector can have the E1-coding sequences removed. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Generation and propagation of replication deficient adenovirus vectors can be performed with helper cell lines. One unique helper cell line, designated 293, was transformed from human embryonic kidney cells by Ad5 DNA fragments and constitutively expresses E1 proteins (Graham et al., 1977). Since the E3 region is dispensable from the adenovirus genome (Jones and Shenk, 1978), adenovirus vectors, with the help of 293 cells, carry foreign DNA in either the E1, the E3, or both regions (Graham and Prevec, 1991).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, a particular helper cell line is 293.

Methods for producing recombinant adenovirus are known in the art, such as U.S. Pat. No. 6,740,320, which is incorporated herein by reference. Also, Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) are employed as follows. A cell inoculum, resuspended in 5 ml of medium, is added to the carrier (50 ml) in a 250 ml Erlenmeyer flask and left stationary, with occasional agitation, for 1 to 4 hours. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 hours.

c. Retroviral Vector

Additionally, the chimeric CD40L polypeptide and/or coronavirus antigen may be encoded by a retroviral vector. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

d. Adeno-Associated Viral Vector

Adeno-associated virus (AAV) is an attractive vector system for use in the present disclosure as it has a high frequency of integration and it can infect nondividing cells, thus making it useful for delivery of genes into mammalian cells (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. Nos. 5,139,941 and 4,797,368.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

e. Coronavirus Vectors

Coronaviruses are positive-sense, single-stranded RNA viruses comprised of four genera: alphacoronavirus, betacoronavirus, gammacoronavirus, and deltacoronavirus. SARS-CoV-1 and SARS-CoV-2 are betacoronaviruses. Coronavirus encode multiple viral proteins, including four major structural proteins: Spike (S), membrane (M), nucleocapsis (N), and envelope (E). Common human coronaviruses, including both alphacoronavirus and betacoronavirus strains are associated with the mild to moderate upper respiratory illnesses, like the common cold. More severe acquired respiratory syndrome illness are caused by MERS-CoV, SARS-CoV-1, and SARS-CoV-2. Coronavirus genomes are relatively large of around 30 kb and may be genetically modified by recombinant genetic manipulation known in the art for removal or inclusion of viral or extraneous genetic material.

Recombinant coronavirus vectors can be generated to contain targeted genetic modifications or for heterologous gene expression. In some embodiments, a recombinant coronavirus vector is made to express a chimeric CD40L. Methods for generating recombinant coronavirus vectors are described (Eriksson et al., 2008, Methods Mol Biol. vol. 454: 237-54). A recombinant coronavirus vector modified to express a chimeric CD40L could be used as a vaccine.

f. Other Viral Vectors

Other viral vectors may be employed as constructs in the present disclosure. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al., 1997).

In further embodiments, the nucleic acid encoding chimeric CD40L and/or coronavirus antigen is housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope (Neda et al., J Biol Chem 1991 vol 4: 14143-46). This modification can permit the specific infection of hepatocytes via sialoglycoprotein receptors.

For example, targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present invention includes constitutive, inducible, and tissue-specific promoters.

a. Promoter/Enhancers

Expression constructs comprise a promoter to drive expression of the polypeptides encoded by the construct. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. For example, in the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

b. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) (Minskaia and Ryan, 2013).

c. Origin of Replication

In order to propagate a vector in a host cell, it may contain one or more origins of replication sites (often termed “ori”), for example, a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

3. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Nucleic Acid Delivery

In addition to viral delivery of the nucleic acids encoding chimeric CD40L and/or coronavirus antigen, the following are additional methods of recombinant gene delivery to a given host cell and are thus considered in the present disclosure.

Introduction of a nucleic acid, such as DNA or RNA, may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Electroporation

In certain particular embodiments of the present disclosure, the gene construct is introduced into target hyperproliferative cells via electroporation. Electroporation involves the exposure of cells (or tissues) and DNA (or a DNA complex) to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for hyperproliferative cells from different sources may be optimized. One may particularly wish to optimize such parameters as the voltage, the capacitance, the time and the electroporation media composition. The execution of other routine adjustments will be known to those of skill in the art. See e.g., Hoffman, 1999; Heller et al., 1996.

2. Lipid-Mediated Transformation

In a further embodiment, the chimeric CD40L and/or coronavirus antigen may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Feigner et al., 1987) and in vivo gene transfer (Zhu el al., 1993; Philip et al., 1993; Solodin et al., 1995; Liu et al., 1995; Thierry et al., 1995; Tsukamoto et al., 1995; Aksentij evich et al., 1996).

Advances in lipid formulations have improved the efficiency of gene transfer in vivo (Templeton et al. 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation forms unique structure termed a “sandwich liposome”. This formulation is reported to “sandwich” DNA between an invaginated bi-layer or ‘vase’ structure. Beneficial characteristics of these lipid structures include a positive ρ, colloidal stabilization by cholesterol, two dimensional DNA packing and increased serum stability. Patent Application Nos. 60/135,818 and 60/133,116 discuss formulations that may be used with the present invention.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

### E. Compositions of Coronavirus Vaccine and/or Chimeric CD40L Polypeptide

In certain embodiments, a coronavirus vaccine is administered at or near the same time as a chimeric CD40L polypeptide or an expression construct encoding a chimeric CD40L polypeptide. In some embodiments, the coronavirus vaccine is included in the same pharmaceutical formulation as the chimeric CD40L polypeptide or an expression construct encoding the CD40L polypeptide. The coronavirus vaccine can comprise a coronavirus antigen, such as coronavirus spike protein. Alternatively, the coronavirus vaccine can comprise inactivated or attenuated coronavirus particles, particularly, the coronavirus particles can be inactivated or attenuated SARS-CoV-1 particles or inactivated or attenuated SARS-CoV-2 particles. In yet another embodiment, the coronavirus vaccine can comprise an expression construct that encodes a coronavirus antigen, such as coronavirus spike protein. Preferably, when the coronavirus vaccine comprises an expression construct, the expression construct encodes a coronavirus spike protein that is, or is substantially homologous to, the coronavirus spike protein for SARS-CoV-1 (SEQ ID NO. 26) or SARS-CoV-2 (SEQ ID NO. 28).

While purified polypeptides or viral vectors of the invention can be administered as isolated agents, it is preferable to administer these viral vectors as part of a pharmaceutical composition. The invention thus further provides compositions comprising a coronavirus vaccine and chimeric CD40L polypeptide or an expression construct encoding a chimeric CD40L polypeptide in association with at least one pharmaceutically acceptable carrier. The compositions administered in accordance with the methods of the invention can be formulated according to known methods for preparing pharmaceutically useful compositions. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions, which may include suspending agents and thickening agents. The formulations may be presented in single-dose or multi-dose containers, for example sealed ampoules and vials, and may be stored in a freeze dried (lyophilized) condition requiring only the condition of the sterile liquid carrier, for example, water for injections, prior to use.

The route of administration for compositions of the invention can be oral, nasal, topical, or injection (including infusion). In embodiments where the coronavirus vaccine and chimeric CD40L are administered separately, the route of administration for each can be the same or different, e.g., the chimeric CD40L polypeptide could be administered by a nasal route of administration substantially contemporaneously to administration of the coronavirus vaccine by injection. For injectable routes of administration, the injection can be subcutaneous, intradermal, intramuscular, intravenous, intratracheal, or intraperitoneal.

The dose level, frequency of dosing, duration of dosing and other aspects of administration of compositions according to the invention may be optimized in accordance with the patient's clinical presentation, weight, and other aspects of clinical care. For compositions comprising one or more viral vectors, each dose may comprise approximately 1e5 to 1e12 viral particles (vp) per vector, and preferable doses from 1e8 to 1e10 vp/vector. For compositions comprising purified polypeptides, each dose may comprise approximately 100 ng to 1 mg of each polypeptide included in such composition, and preferable doses from 1 μg to 100 μg.

### Materials and Methods

### Example 1. Enhanced Immunity Based on Administering Chimeric CD40L and Coronavirus Antigen as Measured by ELISPOT

Balb/c female mice 6-8 weeks old were assigned to the following vaccination groups: Control (phosphate buffered saline); purified recombinant SARS-CoV-1 spike protein (20 ug/dose); MemVax (1e10 viral particles/dose); purified recombinant SARS-CoV-1 spike protein (20 ug/dose) plus MemVax (1e10 viral particles/dose). Mice (n=5 group) were vaccinated by intramuscular injection on days 0 and 15.

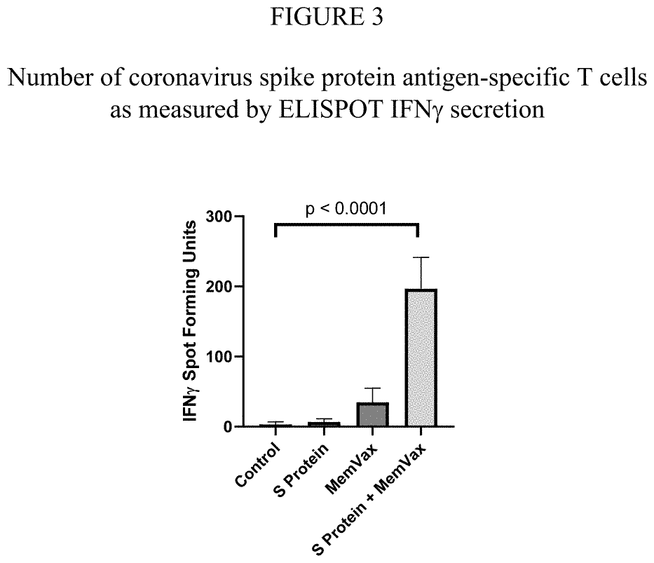

Splenocytes were collected from mice at day 29 and anti-spike protein specific cellular responses were measured by IFNγ ELISPOT. 96-well ELISPOT plates were coated with anti-mouse IFNγ capture antibody followed by plating of splenocytes plus an overlapping 15-mer spike protein peptide mix spanning the entirety of the SARS-CoV-1 spike protein. Following overnight incubation to allow for cellular activation, plates were washed and IFNγ bound cytokine was detected with primary biotinylated anti-IFNγ detection antibody plus secondary streptavidin-horseradish peroxidase antibody. HRP substrate was developed and spots quantitated by microscopy. The results of this assay are shown in FIG. 3 and below in Table 1.

TABLE 1
Number of coronavirus spike protein antigen-specific
T cells as measured by ELISPOT IFNγ secretion

| | IFNγ Spot Forming | Standard | P Value |
|---|---|---|---|
| Vaccine | Units (Mean) | Deviation | (vs Control) |
| Control | 3.0 | 3.9 | NA |
| S Protein | 6.6 | 4.6 | 0.99 |
| MemVax | 34.6 | 20.2 | 0.14 |
| S Protein + MemVax | 196.6 | 44.8 | <0.0001 |

As shown, co-administration of MemVax with the SARS-CoV-1 spike protein generated significant T-cell specific anti-spike protein antigen responses that were not significantly generated with only vaccination using the coronavirus spike protein.

### Example 2. Enhanced Immunity Based on Administering Chimeric CD40L and Coronavirus Antigen as Measured by ELISA

Balb/c female mice 6-8 weeks old were assigned to the following vaccination groups: Control (phosphate buffered saline); purified recombinant SARS-CoV-1 spike protein (20 ug/dose); MemVax (1e10 viral particles/dose); purified recombinant SARS-CoV-1 spike protein (20 ug/dose) plus MemVax (1e10 viral particles/dose). Mice (n=5 group) were vaccinated by intramuscular injection on days 0 and 15.

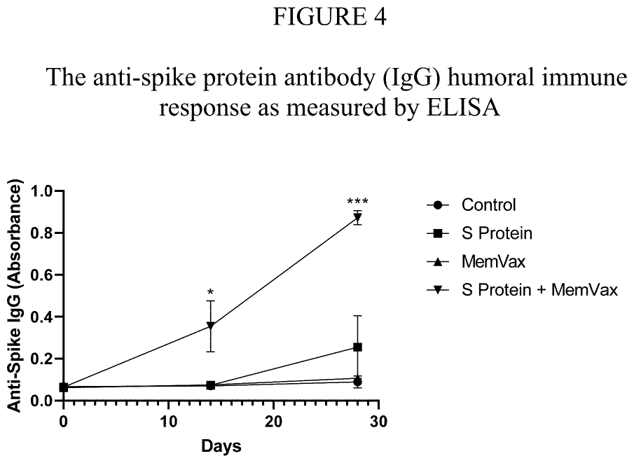

Sera were collected from mice pre-vaccination (day 0), day 14, and day 28. Anti-spike protein IgG antibody was measured in sera by ELISA. Recombinant SARS-CoV-1 spike protein was immobilized onto the surface of the 96-well microtiter plate and then blocked with blocking buffer. 1:200 diluted sera were added to allow any anti-spike protein antibodies to complex. Anti-IgG binding antibodies were detected using a horseradish peroxidase conjugated anti-mouse IgG antibody and tetramethylbenzidine (TMB) chromogenic substrate development and absorbance measurement at 450 nm with a 96-well plate reader. The results of this assay are shown in FIG. 4 and Table 2 below.

TABLE 2
Anti-spike protein antibody (IgG) humoral immune response as measured by ELISA

| | Anti-Spike IgG (Absorbance ± standard deviation) | | | | | |
|---|---|---|---|---|---|---|
| | | P Value | | P Value | | P Value |
| Vaccine | Day 0 | (vs Control) | Day 14 | (vs Control) | Day 28 | (vs Control) |
| Control | 0.067 ± 0.007 | NA | 0.070 ± 0.006 | NA | 0.090 ± 0.029 | NA |
| S Protein | 0.065 ± 0.006 | 0.89 | 0.074 ± 0.017 | 0.94 | 0.256 ± 0.150 | 0.15 |
| MemVax | 0.062 ± 0.004 | 0.43 | 0.076 ± 0.107 | 0.30 | 0.107 ± 0.012 | 0.51 |
| S Protein + | 0.064 ± 0.007 | 0.88 | 0.355 ± 0.122 | 0.01 | 0.872 ± 0.034 | <0.0001 |
| MemVax | | | | | | |

The vaccination group that received coronavirus spike protein with MemVax was the only group able to generate significant anti-spike protein antibody responses following vaccination. The vaccination group that received coronavirus spike protein with MemVax was also able to generate significant anti-spike protein antibody responses after only a single vaccination dose. Moreover, a second vaccine dose of coronavirus spike protein with MemVax was capable of boosting the anti-spike protein antibody response over a single dose vaccination.

All of the methods disclosed and claimed herein can be made and executed without undue experimentation in light of the present disclosure. While the compositions and methods of this invention have been described in terms of particular embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
a coronavirus vaccine; and
at least one of a chimeric CD40L polypeptide or a nucleic acid encoding a chimeric CD40L polypeptide.

2. The composition of claim 1, wherein the CD40L polypeptide is selected from the group consisting of ISF30, ISF31, ISF32, ISF33, ISF34, ISF35, ISF36, ISF37, ISF38, ISF39, ISF40, and ISF41.

3. The composition of claim 2, wherein the chimeric CD40L polypeptide or nucleic acid encoding a chimeric CD40L polypeptide is ISF35.

4. The composition of claim 1, wherein the nucleic acid encoding a chimeric CD40L polypeptide comprises a vector.

5. The composition of claim 4, wherein the vector is a DNA or RNA vector.

6. The composition of claim 5, wherein the vector is a viral vector or plasmid DNA vector.

7. The composition of claim 6, wherein the viral vector is selected from the group consisting of adenoviruses, poxviruses, alphaviruses, arenaviruses, flaviviruses, rhabdoviruses, retroviruses, lentiviruses, herpesviruses, paramyxoviruses, coronaviruses, and picornaviruses.

8. The composition of claim 7, wherein the viral vector is an adenovirus vector.

9. The composition of claim 1, wherein the coronavirus vaccine comprises an expression construct encoding a coronavirus antigen.

10. The composition of claim 9, wherein the coronavirus antigen is a coronavirus spike protein for SARS-CoV-1 or SARS-CoV-2.

11. The composition of claim 1, wherein the coronavirus vaccine comprises a coronavirus antigen.

12. The composition of claim 11, wherein the coronavirus antigen comprises a purified polypeptide.

13. The composition of claim 12, wherein the purified polypeptide is a coronavirus spike protein.

14. The composition of claim 11, wherein the coronavirus antigen comprises inactivated coronavirus particles.

15. The composition of claim 1, which is suitable for administration to a human subject or animal subject.

16. The composition of claim 9, wherein the nucleic acid encoding a chimeric CD40L polypeptide and the coronavirus antigen are encoded from the same expression vector.

17. A method for enhancing immunity comprising:
administering to a human or animal an effective amount of the composition according to claim 1.

18. The method according to claim 17, wherein administering an effective amount of the composition according to claim 1 comprises a route of administration selected from the group consisting of oral, nasal, topical, and injection.

19. The method according to claim 18, wherein the route of administration is injection and selected from the group consisting of subcutaneous, intradermal, intramuscular, intravenous, intratracheal, and intraperitoneal injection.

20. A method of enhancing immunity comprising:
administering a pharmaceutical formulation comprising a coronavirus vaccine; and
administering a pharmaceutical formulation comprising at least one of a chimeric CD40L polypeptide or a nucleic acid encoding a chimeric CD40L polypeptide.

21. The method of claim 20, wherein the step of administering a pharmaceutical formulation comprising at least one of a chimeric CD40L polypeptide or a nucleic acid encoding a chimeric CD40L polypeptide is performed at approximately the same time as the step of administering a pharmaceutical formulation comprising a coronavirus vaccine.

22. A method of enhancing immunity comprising:
admixing a pharmaceutical formulation comprising a coronavirus vaccine with a pharmaceutical formulation comprising at least one of a chimeric CD40L polypeptide or a nucleic acid encoding a chimeric CD40L polypeptide;
administering the admixed formulations to a person or animal.

23. The method of claim 22, wherein the admixing step occurs just prior to the administering step.

24. A method of enhancing immunity comprising: administering a pharmaceutical formulation comprising a coronavirus vaccine and one of: a chimeric CD40L polypeptide or a nucleic acid construct encoding a chimeric CD40L polypeptide.

25. A kit for administration to a person or animal comprising:
a pharmaceutical formulation comprising a coronavirus vaccine; and
a pharmaceutical formulation comprising at least one of a chimeric CD40L polypeptide or a nucleic acid encoding a chimeric CD40L polypeptide.

26. The method of claim 20, wherein the pharmaceutical formulation that comprises a coronavirus vaccine is a viral expression vector that encodes a coronavirus antigen, wherein an amount of viral particles in the formulation is in the range of 1e5 to 1e12 viral particles.

27. The method of claim 26, wherein the amount of viral particles is in the range of 1e8 to 1e11 viral particles.

28. The method of claim 26, wherein the amount of viral particles is 1e10 viral particles.

29. The method of claim 20, wherein the pharmaceutical formulation that comprises at least one of a chimeric CD40L polypeptide or a nucleic acid encoding a chimeric CD40L polypeptide comprises a viral expression vector that encodes a chimeric CD40L polypeptide wherein an amount of viral particles in the formulation is in the range of 1e5 to 1e12 viral particles.

30. The method of claim 29, wherein the amount of viral particles is in the range of 1e8 to 1e11 viral particles.

31. The method of claim 29, wherein the amount of viral particles is 1e10 viral particles.

32. The method of claim 20, wherein the coronavirus vaccine comprises a purified coronavirus spike protein.

33. The method of claim 32, wherein the pharmaceutical formulation of a coronavirus vaccine comprises a coronavirus spike protein in an amount in the range of 1 microgram to 100 micrograms.

34. The method of claim 30, wherein the amount of coronavirus spike protein is 20 micrograms.

35. The method of claim 20, wherein the at least one of a chimeric CD40L polypeptide or a nucleic acid encoding a chimeric CD40L polypeptide is a chimeric CD40L polypeptide and said polypeptide is present in an amount in the range of 1 microgram to 100 micrograms.

36. The method of claim 35, wherein the amount of the chimeric CD40L polypeptide is 20 micrograms.

37. A composition comprising:
a pharmaceutical formulation of a coronavirus vaccine; and
a pharmaceutical formulation of at least one of a chimeric CD40L polypeptide or a nucleic acid encoding a chimeric CD40L polypeptide.

38. The composition of claim 37, wherein the pharmaceutical formulation of a coronavirus vaccine comprises a viral expression vector that encodes a coronavirus antigen, wherein an amount of viral particles in the formulation is in the range of 1e5 to 1e12 viral particles.

39. The composition of claim 37, wherein the pharmaceutical formulation that comprises at least one of a chimeric CD40L polypeptide or a nucleic acid encoding a chimeric CD40L polypeptide comprises a viral expression vector that encodes a chimeric CD40L polypeptide, wherein an amount of viral particles in the formulation is in the range of 1e5 to 1e12 viral particles.

40. The composition of claim 37, wherein the pharmaceutical formulation of a coronavirus vaccine comprises a coronavirus spike protein.

41. The composition of claim 40, wherein the amount of coronavirus spike protein in the pharmaceutical formulation is in a range of 1 to 100 micrograms.

42. The composition of claim 37, wherein the at least one of a chimeric CD40L polypeptide or a nucleic acid encoding a chimeric CD40L polypeptide is a chimeric CD40L polypeptide.

43. The composition of claim 42, wherein the amount of the chimeric CD40L polypeptide is in a range of 1 to 100 micrograms.
